(19) United States
(12) Patent Application Publication
(10) Pub. No.: US 2006/0040905 A1
(43) Pub. Date: Feb. 23, 2006

(54) Formulation of nefopam and its use in the treatment of pain

(76) Inventor: Michael Lyne, Essex (GB)

Correspondence Address: SALIWANCHIK LLOYD & SALIWANCHIK;A PROFESSIONAL ASSOCIATION, PO BOX 142950, GAINESVILLE, FL, 32614-2950, US

(21) Appl. No.: 10/517,882

(22) PCT Filed: Jun. 17, 2003
(86) PCT No.: PCT/GB03/02618

§ 371 Date: Aug. 17, 2005

(30) Foreign Application Priority Data
Jun. 17, 2002 (GB) 0213869.1

(51) Int. Cl. A61K 31/33

(52) U.S. Cl. 514/183

(57) ABSTRACT

(+)-Nefopam is formulated for intranasal administration, for use in the treatment of pain.

### FIELD OF THE INVENTION

This invention relates to a new formulation of nefopam, and to its use in the treatment of pain.

### BACKGROUND OF THE INVENTION

Nefopam is a centrally acting non-narcotic analgesic not structurally related to other analgesics. Nefopam has been shown to induce antinociception in animal models of pain and in humans (reviewed in Heel et al., Drugs 19(4): 249-67, 1980). However, nefopam is not active in the mouse tail-flick test, the hot plate test or the Randall-Selitto pressure test in rats (Conway and Mitchell, Arch. Int. Pharmacodyn. Ther. 226(1): 156-71, 1977), suggesting that its analgesic mechanism is not opiate-like or anti-inflammatory in nature. Nefopam's antinociception is not blocked by nalaxone, further suggesting that its analgesic action is not through opiate receptors.

In vitro and in vivo studies with nefopam enantiomers have shown that (+)-nefopam has more potent analgesic and dopamine-, norepinephrine- and serotonin-uptake inhibitory properties than (−)-nefopam, with the order of potency given as (+)-nefopam>(±)-nefopam>(−)-nefopam (Fasmer et al., J. Pharm. Pharmacol. 42(6): 437-8, 1987; Rosland and Hole, J. Pharm. Pharmacol. 42(6): 437-8, 1990; Mather et al., Chirality 12(3): 153-9, 2000). Mather et al. (2000) conclude that “ . . . there is currently no compelling rationale to justify administering or monitoring individual enantiomers [of nefopam]”.

Nefopam has also been shown to be opiate-sparing when given with morphine in trials of patient-controlled analgesia (Mimoz et al., Anaesthesia 56(6): 520-5, 2001).

Conventional release preparations of nefopam have been commercially available for many years, for use in treating moderate to severe pain. However, the short elimination half-life of nefopam (four hours) means that it is difficult to maintain analgesic efficacy over the normal dosing period (three times daily). Dose escalation of nefopam brings about an increase in the frequency of adverse drug reactions associated with the analgesic, and adverse effects on pulse and blood pressure have been observed following parenteral delivery of therapeutic doses of nefopam (Heel et al., 1980). Chronotropic and ionotropic effects on the heart are not present when nefopam is administered orally (Bhatt et al., Br. J. Clin. Pharmacol. 11(2): 209-11, 1981).

### SUMMARY OF THE INVENTION

According to the present invention, pain such as acute, chronic or neuropathic pain (including, but not limited to, pain associated with cancer, surgery, arthritis, dental surgery, painful neuropathies, trauma, musculo-skeletal injury or disease, and visceral diseases) and migraine headache in mammals, can be treated by the use of (+)-nefopam in a novel formulation, i.e. for intranasal administration.

### DESCRIPTION OF PREFFERRED EMBODIMENTS

The active agent may be in the form of the free base or any pharmaceutically acceptable salt, e.g. the hydrochloride, or in the form of a metabolite or prodrug. Such forms are known to those of ordinary skill in the art.

Nefopam has suitable characteristics for formulation in a composition intended for intranasal administration. It has a low molecular weight, is highly soluble and stable in solution across a wide pH range (4-7) including pH 5.5-6.5 which may be optimal for nasal absorption. Nefopam may thus be rapidly and completely absorbed from the nasal cavity and provide the rapid onset of action required to bring pain relief.

In addition, it has been determined that nefopam demonstrates no cytotoxicity, even at high concentrations (>5 mM), against a nasal epithelial cell-line (RPMI 2650). Nefopam should not irritate the nasal mucosa following nasal delivery in man.

For use in the invention, a medicament may comprise components that are known for the purpose. Intranasal administration of nefopam avoids first-pass metabolism. Nasal delivery introduces significant concentrations of (+)-nefopam to the CNS, while reducing side-effects. In this context, a typical daily dose is less than 60 mg, e.g. 1 to 50 mg, (+)-nefopam.

In particular, it is of benefit to administer nefopam in a manner that reduces peripheral exposure to vascular smooth muscle (minimise effect on vascular tone), while maximising the concentrations in the CNS (maximise analgesia). This may be done by nasal delivery, reducing systemic load, while maximising the concentration of drug in the CNS. By way of example only, a composition for intranasal delivery comprises, in addition to nefopam, one or more of a solubility enhancer such as propylene glycol, a humectant such as mannitol, a buffer and water. Mucoadhesive agents and penetration enhancers may also be used. Such agents and enhancers are known to those skilled in the art.

It will often be advantageous to use nefopam in combination with another drug used for pain therapy. Such another drug may be an opiate or a non-opiate such as baclofen. Especially for the treatment of neuropathic pain, coadministration with gabapentin is preferred. Other compounds that may be used include acetaminophen, a non-steroidal anti-inflammatory drug, a narcotic analgesic, a local anaesthetic, an NMDA antagonist, a neuroleptic agent, an anti-convulsant, an anti-spasmodic, an anti-depressant or a muscle relaxant.

The following Example illustrates the invention.

### EXAMPLE

In the following composition, 1-10 mg nefopam is included in 100 μl of:

| Excipient: | % w/w | |
|---|---|---|
| Benzalkonium chloride | 0.02 | preservative |
| Sorbitol | 15 | humectant |
| Hydroxyethylcellulose | 0.25 | mucoadhesive agent |
| HNa₂PO₄ (0.2 M) | 35.7 | |
| Citric Acid (0.1 M) | 14.1 | |
| Deionised Water | 34.9 | |
| Buffer | to pH 6.5 | |

Stability of nefopam with all the excipients individually has been demonstrated following 4 weeks incubation at both 25° C. and 50° C.

1. (canceled)

2. A composition comprising (+)-nefopam, suitable for intranasal administration.

3. The composition, according to claim 2, which comprises one or mucoadhesive agent, a solubility enhancer, a humectant, a buffer and water.

4. A method for treating pain wherein said method comprises intransasal administration, to an individual in need of such treatment, of (+)-nefopam.
